(19) United States
(12) Patent Application Publication
(10) Pub. No.: US 2007/0068530 A1
(43) Pub. Date: Mar. 29, 2007

(54) Secretion clearing ventilation catheter and airway management system

(76) Inventor: John A. Pacey, Vancouver (CA)

Correspondence Address: IPSOLON LLP, 111 SW COLUMBIA, SUITE 710, PORTLAND, OR, 97201, US

(21) Appl. No.: 11/285,743

(22) Filed: Nov. 21, 2005

Related U.S. Application Data
(60) Provisional application No. 60/629,074, filed on Nov. 19, 2004.

(51) Int. Cl. A62B 9/06; A61M 16/00

(52) U.S. Cl. 128/207.14

(57) ABSTRACT

A pulmonary secretion clearing ventilation catheter and related airway management system is disclosed. The ventilation catheter has a double lumen portion which each lumen of the double lumen portion operably secured to an airway management system so that inspiratory fluid (air/oxygen mixtures, with or without added water vapor) is delivered to the distal end of the ventilation catheter through one of the two lumens and expired inspiratory fluid, pulmonary secretions, and pulmonary fluids are removed from the patent through the other lumen. The expiratory fluid pathway preferably includes a secretion collection system for removing the pulmonary secretions and the like from the pathway, thereby improving operation and safety of the system. An improved inflatable cuff is also disclosed that permits a slight air leakage in the pneumatic seal between the patient's lungs and the environment, thereby facilitating removal of pulmonary secretions from the patient's lungs.

### REFERENCE TO RELATED APPLICATIONS

This application claims priority to U.S. Provisional Application No. 60/629,074, filed on Nov. 19, 2004.

### FIELD OF THE INVENTION

This invention relates to ventilation catheters commonly known as endotracheal tubes, which are used for intra-tracheal ventilation and the like.

### BACKGROUND

The traditional field of airway management includes a process of controlled ventilation that usually uses a mechanical ventilating machine to deliver a predetermined amount of inspiratory fluid, which is usually an air/oxygen gas mixture, with or without added water vapor, to the lungs of a patient on a predetermined cycle. Usually, the ventilating machine cycles between delivering relatively high-pressure inspiratory fluid via a delivery system to the patient's lungs for a short time, and then reducing the pressure in the delivery system for a short time so that used inspiratory fluid within the patient's lungs is expelled. The ventilating machine repeats this cycle of delivering new inspiratory fluid to and then expelling used inspiratory fluid from a patient's lungs, thereby ensuring proper oxygenation of a patient during times when they are unable to breath on their own.

More recently, airway management systems have evolved to permit oxygenation of a patient using oxygenated liquids and/or using a non-cyclic process involving a continuous flow of oxygenated liquids to a patent's lungs while simultaneously maintaining a continuous flow of used fluids from the patient's lungs. An example of these types of systems can be found in U.S. Pat. No. 5,706,830 to Parker.

A ventilation catheter, which is often referred to as an endotracheal tube, is commonly used by both traditional and these more recent airway management systems to deliver inspiratory fluid to the patient's lungs. Historically, the endotracheal tube has been a single tube, the internal cavity of which is often referred to as a lumen, that has an open distal end. The distal end is inserted through a patient's mouth into the patient's trachea so that the distal end is positioned well past the patient's vocal chords. The opposite end of the endotracheal tube is operably connected to a ventilation machine. Accordingly, inspiratory fluid is provided directly to the lungs through the endotracheal tube and used fluid is removed from the patent's lungs through the same tube.

The endotracheal tube must have a reasonably small cross-section to permit easy insertion and positioning of the tube within a patient's trachea. However, the cross-section must be large enough to allow a sufficient flow of oxygenated fluid therethrough.

To date, efforts to improve the use and operation of endotracheal tubes have focused on solving two problems. First, efforts have focused on improving the security and pneumatic sealing of the endotracheal tube within the trachea. Second, efforts have focused on improving the ability of the endotracheal tube to pneumatically isolate individual lungs and/or bronchial chambers within a lung.

Regarding the first problem, one solution that addresses this issue has been to place an inflatable cuff around the endotracheal tube toward the distal end of the tube. The cuff is deflated during insertion of the tube, and inflated when the tube is properly positioned within the trachea, thereby holding the tube in place and creating a pneumatic seal. An example of these types of cuff structures can be found in FIGS. 1A and 1B of U.S. Pat. No. 6,443,156 to Niklason et al.

While the seal offered by these cuffs reduces the likelihood of a patent's airway being inadvertently contaminated with gastric and pharyngeal fluids, they also seal within a patient's lungs pulmonary secretions and fluids. A typical patient can produces about 200 cubic centimeters to 400 cubic centimeters of pulmonary secretions and fluids a day. The volume of these fluids and secretions tends to increase dramatically if a patent also has a pulmonary infection and/or certain types of cardiac disease.

The usual methods for addressing pulmonary secretion and fluid build-up arising during mechanical ventilation of a patient involve periodic suctioning of the patient's lungs and/or an increased antibiotic treatment to address ancillary infections that arise. Such periodic suctioning increases the risk of damaging a patient's pulmonary system and increases the risk of contaminating a patient's airway during each procedure.

Regarding the second problem, some inventors have attempted to isolate lungs and/or bronchial chambers by providing a plurality of individual lumens within the endotracheal tube. Each tube can have its own pneumatic cuff to allow isolation of particular lungs and/or bronchial tubes. However, each tube operates much like a single lumen tube, by providing both inspiratory fluid to the lung and removing used inspiratory fluid from the lung. These types of structures still allow pulmonary secretions and fluids to build-up in the lungs, and the traditional secretion removal and treatment methods must still be employed. Moreover, the cross section of the endotracheal tube can be rather large, thereby limiting the usefulness of the tube in small airways, such as on children and infants.

### SUMMARY OF THE INVENTION

Accordingly, despite the benefits offered by known ventilation catheters, there is still a need for a compact endotracheal tube that can be easily inserted within a patient's trachea that allows for the easy removal of pneumatic secretions and liquids without the need for periodic auxiliary suctioning and the like. In addition to other benefits described herein, the present invention fulfills these needs.

In one disclosed embodiment, the pulmonary secretion clearing ventilation catheter has a double lumen portion witch each lumen of the double lumen portion operably secured to an airway management system so that inspiratory fluid (air/oxygen mixtures, with or without added water vapor) is delivered to the distal end of the ventilation catheter through one of the two lumens and expired inspiratory fluid, pulmonary secretions, and pulmonary fluids are removed from the patent through the other lumen.

The used inspiratory fluid pathway preferably includes a secretion collection system for removing the pulmonary secretions and the like from the pathway thereby improving operation and safety of the system. In addition, by containing the used inspiratory fluid within the system, rather than releasing it to the environment, the release of potentially airborne infective material from a contagious patent, such as SARS and the like, can be minimized.

An improved cuff can also be used. The cuff encircles the distal end of the ventilation catheter to form a substantially pneumatic seal within the trachea. A small channel is formed along one side of the vent so as to allow a small leakage of air from the lungs of the patient during use to the ventilation catheter. This air leakage facilitates removal of secretions from within the patient's lungs without interfering with the ventilation catheter.

Other advantages and features of the present invention will become clear upon study of the following portion of this specification and drawings.

### BRIEF DESCRIPTION OF THE DRAWINGS

FIG. 1 is an isometric view of a first preferred ventilation catheter having a first inflatable cuff in an inflated configuration in accordance with an embodiment of the present invention and showing a possible installation within a cut-away view of a patient's trachea.

FIG. 2 is a cross sectional view of the ventilation catheter of FIG. 1 taken along line 2-2 of FIG. 1.

FIG. 3 is the ventilation catheter of FIG. 1 showing the first inflatable cuff deflated.

FIG. 4 is an isometric view of a second preferred ventilation catheter having a first inflatable cuff in an inflated configuration in accordance with an embodiment of the present invention.

FIG. 5 is a cross-sectional view of the ventilation catheter of FIG. 4 taken along line 5-5 of FIG. 4.

FIG. 6 is a schematic diagram of an airway management system with a ventilation catheter of FIG. 1 in accordance with an embodiment of the present invention.

FIG. 7 is a schematic diagram of an alternative preferred airway management system in accordance with an embodiment of the present invention showing a possible flow circuit of oxygenated fluid to a patient's lungs.

FIG. 8 is the schematic diagram of FIG. 7 showing a possible discharge circuit from the patient's lungs through the airway management system.

FIG. 9 is a schematic diagram of a third preferred ventilation catheter in accordance with an embodiment of the present invention.

FIG. 10 is the third preferred ventilation catheter of FIG. 8 showing a cuff in a possible deflated position.

FIG. 11 is an isometric view of a fourth preferred ventilation catheter showing a second preferred cuff operably attached thereto.

FIG. 12 is a cross-section view of the fourth preferred ventilation catheter of FIG. 11 taken along line 12-12 of FIG. 11.

FIG. 13 is an isometric view of a fifth preferred ventilation catheter in accordance with an embodiment of the present invention.

### DETAILED DESCRIPTION OF PREFERRED EMBODIMENTS

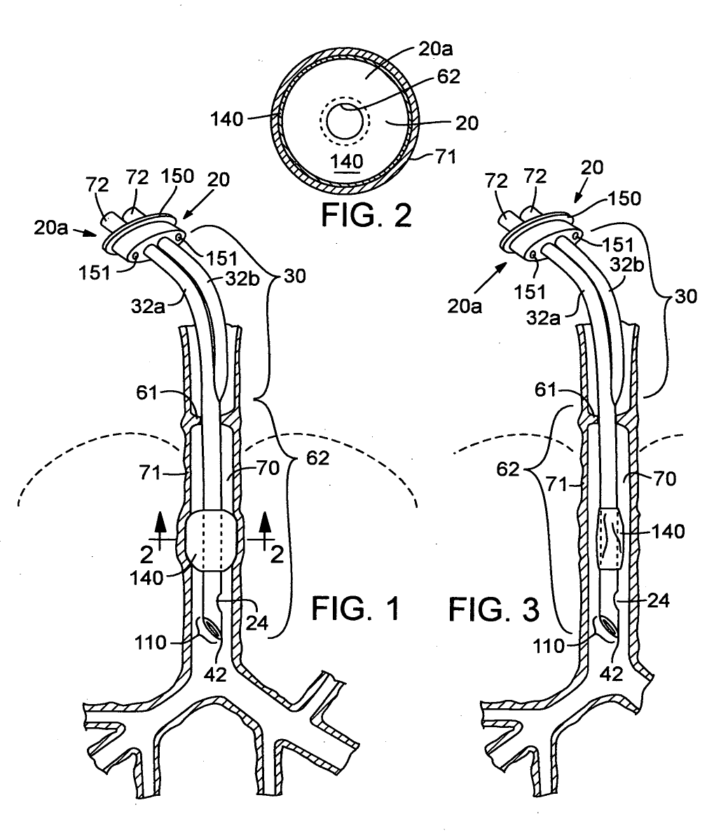

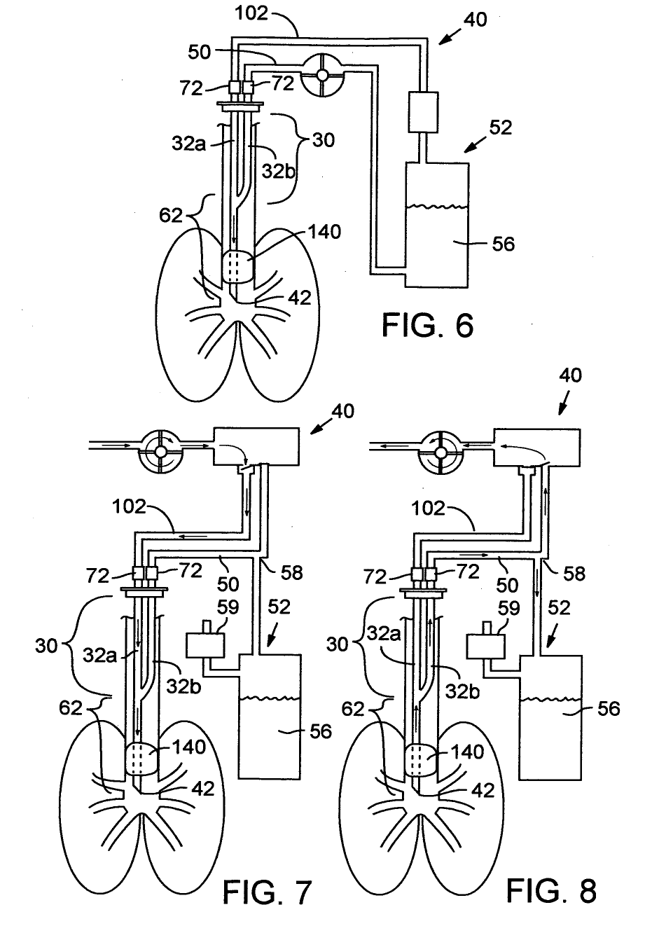

A pulmonary secretion clearing ventilation catheter 20 and related airway management system 22 are disclosed in FIGS. 1-13. In general, the ventilation catheter 20, which is also referred to as an endotracheal tube, extends into the trachea 24 of a patient 26 to provide ventilation. The ventilation catheter 20 has a double lumen portion 30. Each lumen 32a, 32b of the double lumen portion 30 is operably secured to an airway management system 40 (FIGS. 6-8) so that inspiratory fluid (air/oxygen mixtures, with or without added water vapor) is delivered to the distal end 42 of the ventilation catheter 20 through one of the two lumens 32a, 32b (here 32a is shown) and expired inspiratory fluid, pulmonary secretions, and pulmonary fluids are removed from the patent through the other lumen 32a, 32b (here 32b is shown).

Separating the incoming and outgoing inspiratory fluid flow through separate lumens 32a, 32b prevents the fresh incoming inspiratory fluid from becoming blocked or contaminated by inadvertent pulmonary secretions and fluids mixed with the used inspiratory fluid. The used inspiratory fluid pathway 50 (FIGS. 6-8) preferably includes a secretion collection system 52 for removing the pulmonary secretions and the like from the pathway 50, thereby improving operation and safety of the system. In addition, by containing the used inspiratory fluid within the system, rather than releasing it to the environment, the release of potentially airborne infective material from a contagious patent, such as SARS and the like, can be minimized.

Several ventilation catheter embodiments having these basic features are disclosed in this application. In order to reduce undue repetition like elements between these embodiment have like element numbers.

Preferably and referring to FIG. 1, a first preferred ventilation catheter 20a is disclosed. In this embodiment, the double lumen portion 30 is positioned at the pre-pharyngeal/pharynx region of the patient upstream of their vocal chords 61. Each lumen 32a, 32b of the double lumen portion 30 pneumatically joins together to form a single lumen portion 62 that extends toward the distal end 42 of the ventilation catheter 20a. The single lumen portion 62 protrudes into the intratracheal region 70 of the patient. This allows a small diameter single lumen portion 62 to extend past the vocal chords into the trachea of the patent, while still allowing the secretion clearing benefits of the double lumen portion 30. The double lumen portion 30 positioned in the pre-pharyngeal level increases the efficiency of ventilation, reduces unnecessary dead space, eliminates secretions, and permits a smaller diameter intratracheal catheter to be used.

Preferably, each lumen 32a, 32b of the double lumen portion 30 has a proximal end 80 to which are connected conventional adapters 72 for detachable securing to mating connectors on a conventional ventilation system 40 (FIG. 6). One lumen 32a is connected through its connector to a delivery conduit 102 of a conventional ventilation system 100 (FIG. 6). The other lumen 32b is connected to the used inspiratory fluid pathway 50 of the ventilation system 100. The system 100 is controllable for delivering inspiratory fluid at pre-selected flow rates. The inspiratory fluid is thus delivered to the proximal end of lumen 32a at a pressure of about 20 mmHg for a selected period of time. After which, the system is vented in a way to drop the pressure in the system to about 6 mmHg during the expiratory phase of the ventilation cycle. This causes fluid, along with pulmonary secretions and pulmonary fluids to exit the patient through the second lumen 32b.

More preferably, the ventilation system 100 is configured to deliver inspiratory fluid through one lumen 32a of the double lumen portion 30 on inspiration while used inspiratory fluid and secretions are expelled from the patient through the other lumen 32b of the double lumen portion 30 during both inspiration and expiration phases of the ventilation system 40.

The tip 110 of the ventilation catheter 20a is preferably beveled and softened usefully to assist in the passage of the single lumen portion during the intubation of the trachea. The tip is preferably designed to resist the backward bending that might obstruct the airway.

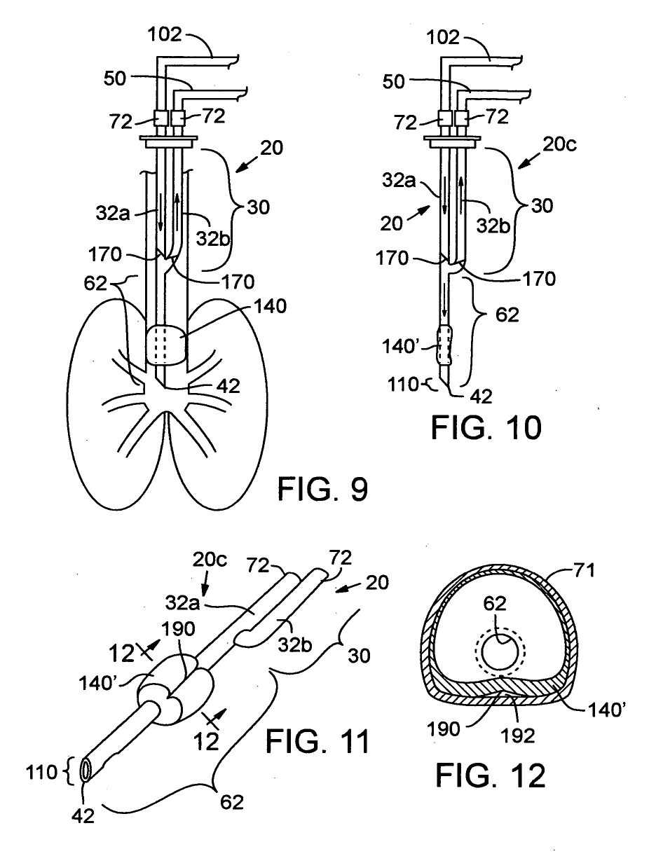

The double lumen portion and single lumen portion of the ventilation catheter are preferably made of a soft, clear medically approved elastomer. If desired and referring to FIG. 9, one-way check-valves 170, such as flapper valves, can be posited within one or both lumens of the double lumen portion to further prevent back-flow through these lumens.

Preferably, a low pressure, inflatable cuff 140 is positioned toward the distal end 42 of the ventilation catheter. The inflatable cuff 140 is preferably made of a thin film of substantially impermeable plastic or the like. The edges of the cuff are bonded to the outer surface of the single lumen portion 62. The cuff 140 is inflated with known means, such as those disclosed in FIGS. 1A and 1B of U.S. Pat. No. 6,443,156, which involves extending a cuff inflation line within the ventilation catheter 20a from the cuff 140 to an auxiliary inflator.

The ventilation catheter 20a is inserted into a patient's trachea 24 when the cuff 140 is deflated as shown in FIG. 3. Then, the cuff 140 is inflated. Once inflated, the cuff 140 expands as shown in FIGS. 1 and 2 to assume the substantially circular cross-sectional shape of the trachea, thereby pneumatically sealing the patient's lungs to the ventilation catheter 20a.

Preferably, a durable, bite-resistant, bite block 150 is secured to the double lumen portion of the ventilation catheter as shown in FIG. 1. Each lumen 32a, 32b of the double lumen portion 30 extends though the bite block 150. More preferably, the bite block 150 is slidably secured to the double lumen portion 30 so as to allow the position of the bite block 150 relative to a patient's teeth and lips to be adjusted as needed by sliding the bite block along the double lumen portion. More preferably, the bite block 150 includes one or more auxiliary holes therethrough or the like that are in parallel with the double lumen portion 30. These holes can be used as needed for endo-bronchial blocking catheters, fiberoptic endoscopes, and the like. Vent holes 151 can also be provided as needed.

Preferably, the ventilation system 40 (FIG. 6) is adapted as shown in FIGS. 7 and 8 to include a secretion collection system 52 in the outflow circuit. The secretion collection system 52 preferably includes a secretion collection chamber 56 in pneumatic communication with the used inspiratory fluid pathway 50 at a diverter 58. As secretions in the used inspiratory fluid pathway 50 pass by the diverter 58, they are directed toward the collection chamber 56 either by gravity, or with the assistance of an auxiliary pump 59.

If desire, one or more ultraviolet light generating bulbs in the desired flow path(s) and the like to provide desired antibacterial activity as needed. Similarly, an appropriate antibacterial/anti-virus filter can be posted within the system to prevent exhaust gasses and the like from being released into the environment.

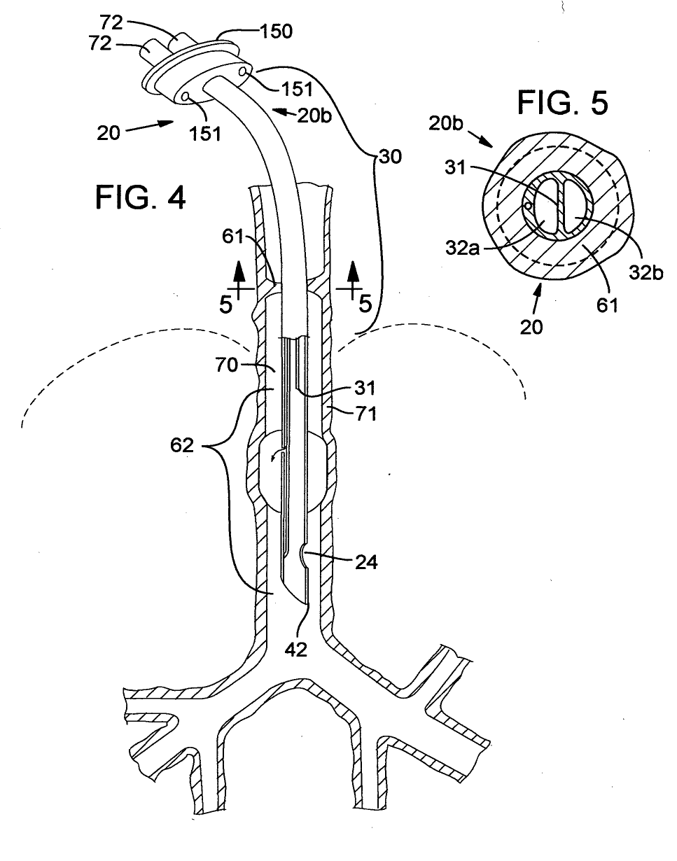

Referring to FIGS. 4 and 5, a second preferred ventilation catheter 20b is shown. In this embodiment, the double lumen portion 30 is formed by a longitudinal wall 31 extending down the middle of a single lumen to define two lumens 32a, 32b. The wall 31 is removed toward the distal end 42 of the ventilation catheter 20b thereby combing the two lumens 32a, 32b into a single lumen portion 62. As with the first preferred ventilation catheter 20a (FIGS. 1-3), oxygenate fluid in this embodiment 20b, is delivered through one lumen 32a to the single lumen portion 62, and used inspiratory fluid is removed from the patient through the second lumen 32b.

Referring to FIGS. 10 and 11, a third preferred ventilation catheter 20c is shown. In this embodiment, the double lumen portion 30 is formed by a single lumen extending down the entire length of the ventilation catheter 20c, thereby defining a substantially straight channel through which to insert auxiliary devices, such as fiberoptic endoscopes, and the like. The second lumen 32b of the double lumen portion 30 intersects the single lumen substantially at a right angle as shown thereby defining the double lumen portion 30 of the ventilation catheter 20c.

As best shown in FIGS. 11 and 12, an alternative preferred lower pressure, inflatable cuff 140′ is positioned toward the distal end 42 of the ventilation catheter 20c. The alternative preferred inflatable cuff 140′ is preferably made of a thin film of substantially impermeable plastic or the like. The edges of the cuff are bonded to the outer surface of the single lumen portion 62, and a recessed channel 190 is provided on one side of the inflated cuff 140′.

The cuff 140′ is inflated with known means, such as those disclosed in FIGS. 1A and 1B of U.S. Pat. No. 6,443,156, which involves extending a cuff inflation line within the ventilation catheter from the cuff to an auxiliary inflator.

The ventilation catheter 20c is inserted into a patient's trachea 24 without the cuff 140′ inflated. Then, the cuff 140′ is inflated. Once inflated, the cuff expands as shown in FIGS. 11 and 12 to assume the substantially circular cross-sectional shape of the trachea 24, thereby substantially pneumatically sealing the patient's lungs to the ventilation catheter. The recessed channel 190 allows a small pneumatic opening 192 between the cuff 140′ and the trachea wall 71, thereby allowing a limited pneumatic leak from the patient's lungs to the environment. Preferably, the channel 190 is sized so as to allow about 10% of the inspiratory fluid delivered through the ventilation catheter 20c to exit through the pneumatic opening 192.

This continuous leaking facilitates secretion clearing of the lungs. Pulmonary secretions and the like travel up the trachea through the pneumatic opening 192 in the cuff 141‘to the patient’s hypopharynx, where they can be easily suctioned way without disruption the ventilation catheter.

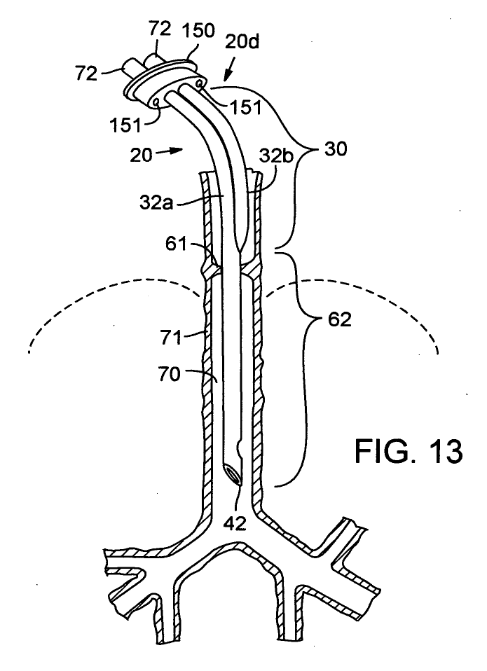

Referring to FIG. 13, a fourth ventilation catheter 20d is disclosed. This catheter is substantially similar to the first disclosed embodiment of FIG. 1, but does not have an inflatable cuff 140 (FIG. 1) operably secured thereto. This cuffless design is particularly useful when working in small tracheas, such as those found in infants and small children.

While the present invention has been described in terms of preferred embodiments, it will be appreciated by one of ordinary skill that the spirit and scope of the invention is not limited to those embodiments. For example, the alternative preferred cuff 140′ (FIGS. 10 & 11) and/or check valves 170 (FIG. 9) could be installed on any disclosed embodiment of the ventilation catheters 20a-d. Accordingly, the scope of the present invention extends to the various modifications and equivalents as defined in the appended claims.

I claim:

1. A ventilation catheter for operably securing to an airway management system that has a new inspiratory fluid path and an expiratory fluid path, said catheter having:
an open distal end for allowing new inspiratory fluid from the airway management system to flow into a patient and used inspiratory fluid from the patent to flow back to the airway management system;
a double lumen portion defining a first lumen and a second lumen, said double lumen portion sized to be received within a patient's mouth and extend down the patient's throat;
said first lumen pneumatically connected to the new inspiratory fluid path; and,
said second lumen pneumatically connected to said expiratory fluid path.

2. The ventilation catheter of claim 1, wherein said double lumen portion is formed from a single lumen with an elongate wall extending therethrough, thereby defining said first lumen and said second lumen therein.

3. The ventilation catheter of claim 1, wherein said first lumen and said second lumen pneumatically join together to define a single lumen portion, said single lumen portion positioned toward the distal end of the ventilation catheter.

4. The ventilation catheter of claim 4, wherein said double lumen portion is formed by securing said second lumen substantially perpendicular to said first lumen, thereby defining a substantially straight path from said first lumen through said single lumen portion.

5. The ventilation catheter of claim 2, further including an inflatable cuff operably secured to the single lumen portion.

6. The ventilation catheter of claim 5, wherein said inflatable cuff has an elongate channel along an outer surface, thereby defining an airway leaking channel when the cuff is inflated in the trachea of a patient.

7. The ventilation catheter of claim 6, wherein the airway leaking channel is sized to allow about 10% of the inspiratory fluid flow provided by the airway management system to leak therethrough.

8. The ventilation catheter of claim 1, further including a bite protector slidably secured to the double lumen portion.

9. The ventilation catheter of claim 8, wherein said bite protector includes at least one channel therethrough to permit access to the new inspiratory fluid path within the ventilation catheter.

10. The ventilation catheter of claim 1, wherein said new inspiratory fluid is a gas.

11. The ventilation catheter of claim 3, wherein said double lumen portion is sized to be positioned upstream of the patient's vocal chords with the single lumen portion extending past the patient's vocal chords into the patient's trachea.

12. The ventilation catheter of claim 1, wherein said expiratory fluid path includes a secretion collector for collecting pulmonary secretions and pulmonary fluids collected from the patient through the expiratory fluid path.

13. The ventilation catheter of claim 12, wherein said secretion collector includes a secretion chamber for collecting secretions therein.

14. The ventilation catheter of claim 1, further including a check-valve operably secured to at least one said first lumen and said second lumen to prevent inadvertent pneumatic back flow.

15. The ventilation catheter of claim 14, further including a second check valve operably secured to the other of said at least one said first lumen and said second lumen.

16. The ventilation catheter of claim 1, further including a check valve in the second lumen thereby preventing inadvertent backflow from the expiratory fluid path toward the distal end of the ventilation catheter.
